(19) United States
(12) Patent Application Publication
(10) Pub. No.: US 2005/0037338 A1
(43) Pub. Date: Feb. 17, 2005

(54) Method and means for detection of severe acute respiratory syndrome

(76) Inventor: Kuo-Tang Tseng, Taipei (TW)

Correspondence Address: BRUCE H. TROXELL;SUITE 1404, 5205 LEESBURGE PIKE, FALLS CHURCH, VA, 22041, US

(21) Appl. No.: 10/640,032

(22) Filed: Aug. 14, 2003

(51) Int. Cl. C12Q 1/70; C12Q 1/68; C12P 19/34

(52) U.S. Cl. 435/5; 435/6; 435/91.2

(57) ABSTRACT

A diagnostic means and method of detecting the presence or the absence of severe acute respiratory syndrome coronavirus antibody in a test sample. The means comprises a solid support, an unlabeled recombinant antigen peptide on a first area of the solid support, a labeled recombinant antigen peptide on a second area of the solid support and an internal control antibody peptide on a third area of the solid phase. The first area is located between the second and the third area.

### FIELD OF THE INVENTION

This invention relates to the detection the presence or the absence of SARS antibody in a biological sample.

### BACKGROUND OF THE INVENTION

SARS was proofed to be caused by a new strain of a coronavirus which may have “jumped” from animals to humans in the southern Chinese province of Guangdong.

Coronaviruses are a group of viruses that have a halo or crown-like (corona) appearance when viewed under a microscope. These viruses are a common cause of mild respiratory illness (e.g. colds). Coronaviruses can only survive in the environment for at least several hours.

The illness usually begins with a fever (measured temperature greater than 38.0° C. [>100.4° F.]). The fever is sometimes associated with chills or other symptoms, including headache, general feeling of discomfort, body aches and diarrhoea. Some people also experience mild respiratory symptoms at the outset.

After 2 to 7 days, SARS patients may develop a dry cough that might be accompanied by or progress to the point where insufficient oxygen is getting to the blood. In 10 percent to 20 percent of cases, patients will require mechanical ventilation.

The primary way that SARS appears to spread is by close person-to-person contact. Most cases of SARS have involved people who cared for or lived with someone with SARS, or had direct contact with infectious material (for example, respiratory secretions) from a person who has SARS. Potential ways in which SARS can be spread include touching the skin of other people or objects that are contaminated with infectious droplets and then touching your eye(s), nose, or mouth. This can happen when someone who is sick with SARS coughs or sneezes droplets onto themselves, other people, or nearby surfaces. It also is possible that SARS can be spread more broadly through the air or by other ways that are currently not known.

At present, tests are performed for people suspected of having SARS. These tests are still under development. A blood test 21 days after the acute illness is the WHO recommended test to see if a person was infected with the SARS coronavirus. If a person has a positive test for the SARS corona virus it means they are, or recently were, infected with the virus. Having a negative test by PCR for the SARS coronavirus does not, however, mean that a person is definitely not infected.

With this background in mind a rapid, sensitive and specific detection is imperative.

### SUMMARY AND OBJECTS OF THE INVENTION

The present invention is directed to nucleic acid sequences (oligonucleotides) useful as primers in gene amplification to obtain targeting fragment encoding spike protein. Also, the present invention is directed to a recombinant spike protein purified from the prokaryote transfected by a vector with gene amplification product integrated therein. Further, the present invention is directed to a means and a method of detecting the presence or the absence of severe acute respiratory syndrome coronavirus antibody in a test sample using a rapid, sensitive and accurate way.

One object of the present invention is to provide oligonucleotides which can be used as primers to amplify specific nucleic acid sequences of severe acute respiratory syndrome coronavirus.

Another object of the present invention is to provide a recombinant spike protein purified from the prokaryote transfected by a vector with the amplified nucleic acid sequence incorporated therein.

Another object of the present invention is to provide a rapid, sensitive and accurate method for detecting the presence or the absence of severe acute respiratory syndrome coronavirus antibody suspected of containing in test samples.

A further object of the present invention is to provide a rapid, sensitive and accurate means for detecting the presence or the absence of severe acute respiratory syndrome coronavirus antibody suspected of containing in test samples.

### BRIEF DESCRIPTION OF THE DRAWINGS

The foregoing aspects and many of the attendant advantages of this invention will become more readily appreciated and understood by referencing the following detailed description in conjunction with the accompanying drawings, wherein:

FIG. 1 shows nucleotide sequences of targeting fragment;

FIG. 2 shows amino acid sequences derived from the nucleotide sequences;

FIG. 3 represents the solid support comprises a wicking pad, an analytical membrane, a conjugate pad and a sample pad;

FIG. 4 represents unlabeled recombinant antigen and anti-human antibody immobilized on analytical membrane of the solid support to form respective two bands;

FIG. 5 represents labeled recombinant antigen immobilized on conjugate pad of the solid support to form an area; and

FIG. 6 shows diagnoses of detection.

### BEST MODE FOR CARRYING OUT THE INVENTION

Now, the present invention will be described in detail. In the embodiment of the present invention, the polynucleotide sequences provided herein can be advantageously used as primers for nucleic acid hybridization. As such, it is contemplated that nucleic acid segments that comprise a sequence region of at least about 15 nucleotide long contiguous sequence that has the same sequence as, or is complementary to, a 15 nucleotide long contiguous sequence disclosed herein will find particular utility. Longer contiguous identical or complementary sequences, e.g., those of about 20, 30, 40, 50, 100, 200, 500, 1000, 1200 (including all intermediate lengths) will also be of use in certain embodiments.

This invention uses synthetic oligonucleotide sequences as primers. These sequences can be prepared by well-known chemical procedures, and commercially available DNA synthesizers can also be used. For example, the required sequence can be prepared by the synthesis method described by Beaucage, et al., Tetrahedron letters, 22: 1859-62 (1981). Another method for the synthesis of oligonucleotide on the solid support is described in U.S. Pat. No. 4,458,066. Automated DNA synthesis means can be used such as the DNA synthesizer sold by Applied Biosystems.

The single strand oligonucleotide is represented by the standard letter abbreviations in which the nucleotide are designate as follows: A for adenosine, T for thymidine, G for guanosine, and C for cytosine. These strands are represented in a standard 5′ prime to 3′ prime orientation.

Primer Sequences

| | |
|---|---|
| 5′ TTGGATCCACCGGCCACGGTTTGTGGACCA | SEQ ID NO: 1 |
| 5′ TAGAATTCATTTTGGGTAACTCCAATGCCA | SEQ ID NO: 2 |

### Primer Selection

The primer sequences can be used in the genetic amplification of the SARS cDNA. The primers are designed to hybridize with a region that encodes spike protein of SARS coronavirus. These primers are capable of effectively hybridizing and serving as primers for the thermostable DNA polymerase used in the amplification process. More specifically the primer selection involved the following process.

Complete genome of SARS coronavirus was obtained from NCBI (http://www.ncbi.nlm.nih.gov/). The unique region encoding spike protein was analyzed as potential oligonucleotide primers using software specifically designed for primer selection, NUCIT.TM. (Compuright, Washington Grove, Md.).

More specifically, with the aid of the above discussed selection software, a primer pair was selected: 5′ TTGGATCCACCGGCCACGGTTTGTGGACCA (SEQ ID NO: 1) and 5′ TAGAATTCATTTTGGGTAACTCCAATGCCA (SEQ ID NO: 2), that would encode a 1173 basepair fragment (nt. 23008-24180) of SARS spike protein mRNA.

Reverse transcription and PCR amplification of SARS mRNA with primers selected from sequence information to the spike protein of SARS resulted in a discrete fragment of approximately 1200 base pairs. When SARS RT-PCR fragment was cloned in pGEX-1 vector (Amersham Bioscience), transformed into E. coli (Novagen), and sequenced (f mole TM DNA Sequencing Sytem, Promega, Madision, Wis.) indicated that the insert was a 1173 base pair identical to sequences of SARS (nucleotides 23008-24180) described by Roper et al. (2003).

### Nucleic Acid Amplification Method

The SARS cDNA may be amplified using the polymerase chain reaction (PCR). cDNA to be amplified is distributed in 0.5 ml microfuge tubes using 30 μl of cDNA or adjusting the volume to 30 μl with sterile distilled water. A reaction mixture is prepared in sufficient volume to add 70 μl to each individual reaction tube. Stock reagents for the reaction mixture are mixed in the following proportions for each PCR reaction: 10 μl of 10×PCR buffer (100 mM Tris pH 8.3, 500 mM KCl, 15 mM MgCl₂, 0.1% gelatin) 16 μl of dNTPs (1.25 μM each dATP, dCTP, dGTP, dTTP), 0.2 μM of the primer, and 1 unit of Taq polymerase. The total volume is achieved by addition of sterile distilled water. The Taq polymerase is added to the reaction mixture just before use and is gently mixed, not vortexed. The reaction mixture is then added (70 μl/tube) to the tubes containing target cDNA to be amplified, and the tubes are gently mixed and briefly centrifuged. An overlay of mineral oil, approximately 2 drops, is added to each tube and then the tubes were placed in the thermal cycler. Thirty to thirty-five cycles are generally sufficient for SARS coronaviral cDNA amplification. One cycle consists of 1 minute at 94° C., annealing for 1 minute at 55° C., and primer extension for 2 minutes at 72° C. The first cycle includes a 5 minute incubation at 94° C. to assure complete denaturation. Once amplified, the PCR fragments are isolated by gel electrophoresis and sequenced.

Sequence of the targeting fragment obtained from PCR reaction is represented by the standard letter abbreviations in which the nucleotide are designate as follows: A for adenosine, T for thymidine, G for guanosine, and C for cytosine. These strands are represented in a standard 5′ prime to 3′ prime orientation.

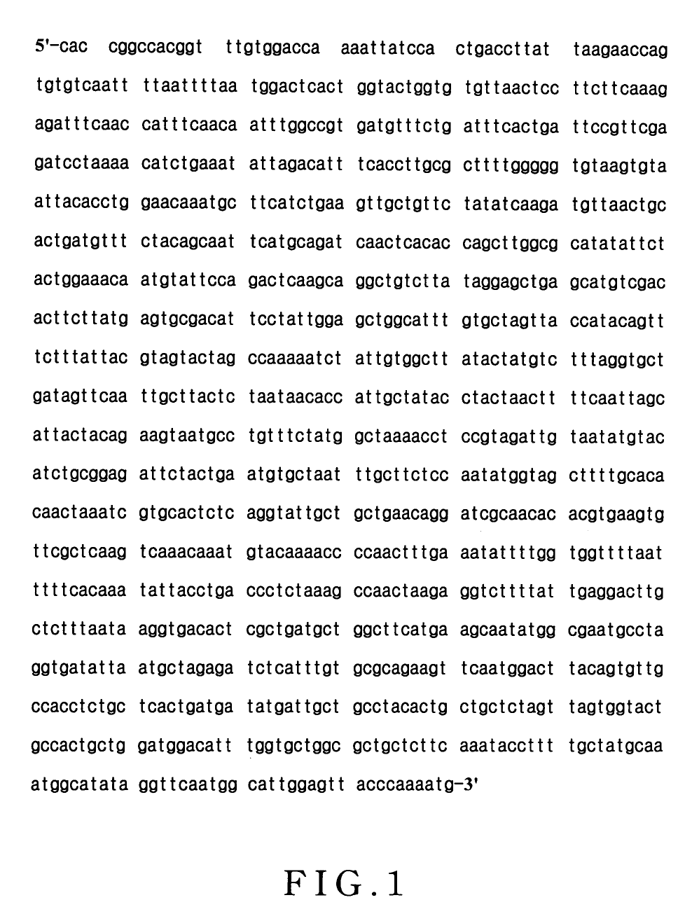

Sequences of targeting fragment (SEQ ID NO: 3) is shown in FIG. 1.
Examples of purification of proteins starting from 500 ml of culture:

The PCR fragment is then integrated into pGEX-1 vector. The strain E. coli RV 308 (Maurer et al., J. Mol. Biol., 1980, 139, 147) transfected by the vector was selected on agar containing ampicillin (100 μg/ml) and tetracycline (8 μg/ml). The strain was inoculated into an Erlenmeyer flask containing 100 ml of TSB culture medium (Tryptic Soy broth, Difco) (30 g/l), supplemented with yeast (Yeast Extract, Difco) (5 g/l), ampicillin (100 μg/ml), tetracycline (8 μg/ml) and tryptophan (100 μg/ml). Incubate at 32° C. for 12 hours with stirring (190 rpm). Transfer the culture into another erlenmeyer flask (5 liters) containing four times the initial volume (400 ml of TSB+yeast+the same antibiotics at the same concentration). When the optical density of the medium (at 550 nm) has reached an O.D. of approximately 1.5, the production of the proteins is induced by adding IAA to the medium to a final concentration of 25 μg/ml. Culturing is stopped after incubation for 5 hours, with stirring (190 rpm) at 32° C. After centrifugation, the bacterial plug is resuspended in a vessel comprising approximately 60 ml of cold TST solution (50 mM Tris HCl, pH 8.0, 200 mM NaCl, 0.05% Tween 20, 0.5 mM EDTA).

A standard sonicator probe (VIBRA-CELL, Somics Mat, USA) is introduced into the vessel. Sonication is carried out at a power of 5 for approximately two minutes. The supernatant of the solution after centrifugation is filtered at 0.45 μm, and passed into a column containing approximately 3 ml of Glutathion-sepharose gel (ST.ANG.HL et al., J. Immunol. Meth., 1989, 124, 43).

The purified proteins are analyzed by SDS-PAGE on a Phast System means (PHARMACIA) or on Mini Protean BIORAD. The gels are visualized by Coomassie Blue. The protein spike, representing more than 95% purity, corresponds well to the expected size with respect to known molecular weight standards. The yield of purified soluble proteins starting from the cytoplasm of E. coli is approximately 50 mg/liter of culture.

In a 2-liter culture, it is possible to obtain 500 to 800 mg of spike proteins per liter of culture under optimum culture conditions.

The spike protein is represented by the standard letter abbreviations in which the peptide are designate as follows: A for alanine, C for cysteine, D for aspartic acid, E for glutamic acid, F for phenylalanine, G for glycine, H for hisitdine, I for isoleucine, K for lysine, L for leucine, M for methionine, N for asparagines, P for proline, Q for glutamine, R for arginine, S for serine, T for threonine, V for valine, W for tryptophan, and Y for tyrosine. The strand is represented in a standard N-terminal to C-terminal orientation.

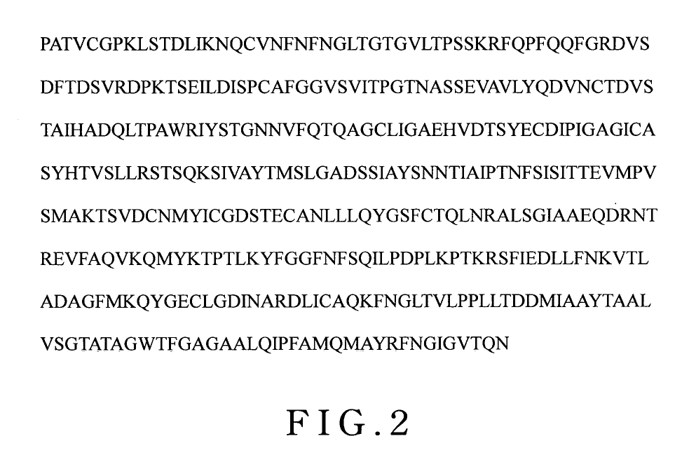

Amino acid sequence (SEQ ID NO: 4) is shown in FIG. 2.
Means and method of detecting anti-SARS coronavirus antibody

I. Means

1. Solid Support (Substrate)

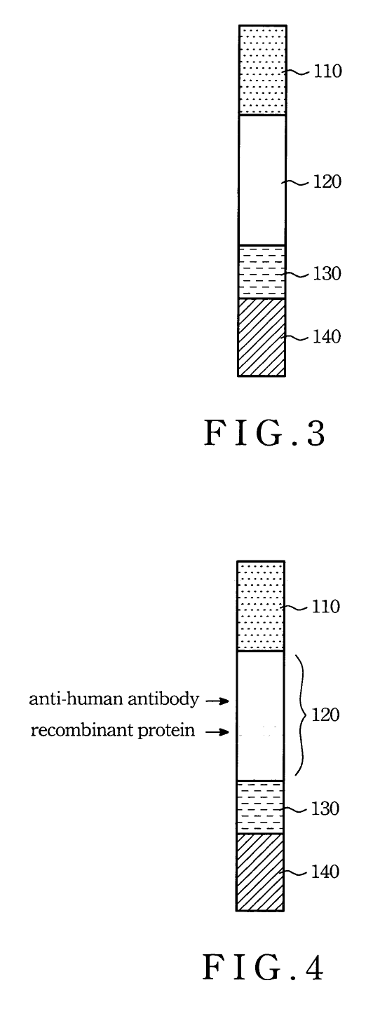

A variety of materials can be used as the solid support of the means. Organic and inorganic polymers, both natural and synthetic, can be used as the solid support. Examples of suitable polymers include polyethylene, polypropylene, polybutylene, poly(4-methylbutylene), butyl rubber, polyesters, polyamides, cellulose and cellulose derivatives (such as cellulose acetate, nitrocellulose and the like), acrylates, methacrylates, vinyl polymers (such as polyvinyl acetate, polyvinyl chloride, polyvinylidene chloride, polyvinyl fluoride, and the like), polystyrene and styrene graft copolymers, rayon, nylon, glass fiber, polyvinylbutyrate, polyformaldehyde, etc. Other materials which can be used as the solid support can be the latexes of the above polymers, silica gel, silicon wafers, filter papers, semi-conductive materials, cermets and the like. In a preferred embodiment of the present invention, compositions of the solid support are assembled as shown in FIG. 3.

Referring to FIG. 3, the solid support 100 comprises a wicking pad 110 (470, Schleicher & Schuell), an analytical membrane 120 (FF85/100, Schleicher & Schuell), a conjugate pad 130 (33GLASS, Schleicher & Schuell) and a sample pad 140 (16-S, Schleicher & Schuell). The material of wicking pad 110 is preferably selected from filter paper, whereas the analytical membrane 120 is selected from nitrocellulose. The conjugate pad 130 and the sample pad 140 comprise glass fiber.

2. Means for Detecting Anti-SARS Coronavirus Antibody

In a preferred embodiment of the present invention, an anti-human antibody such as anti-GST antibody obtained from immunized animal is diluted with PBS, which has a phosphate molarity of 0.01 and a pH of from 6 to 8, to a concentration of 1 μg/μl, to serve as an internal control. The recombinant antigen, i.e. spike protein purified from the E. coli, is diluted with 0.01M PBS as well. Referring to FIG. 4, the diluted anti-human antibody and recombinant protein are sprayed on the analytical membrane 120 to form respective two bands of a concentration of 1 μl/cm by a sprayer (XYZ3000, BioDot). It is noted that the band of anti-human antibody is located between the wicking pad 110 and the band of recombinant antigen.

Next, the solid support 100 with anti-human antibody and recombinant protein immobilized thereon is dipped into 10 mM Tris buffer containing 5% bovine serum albumin (BSA) and 0.1% Tween 20 (pH of the buffer solution is adjusted to 8.0 before use) for blocking. Subsequently, a drying process is carried out.

In a preferred embodiment of the present invention, a biological sample is reacted with a solid support having recombinant spike protein immobilized thereon to serve as an antigen. For preparing the antigen, the recombinant spike protein purified from E. coli is conjugated with and lableled by colloidal gold.

The recombinant protein is added to colloidal gold solution to a concentration of 8 μg/ml and stirred for 15 minutes at room temperature. Thereafter, 200 μl of 10 mM Tris buffer containing 5% BSA is added to 1 ml of the colloidal solution for blocking, and stirred for 30 minutes at room temperature. The solution is then centrifuged at 9000 rpm for 20 minutes at 4° C. It is noted that other detectable labels may also be used, such as an enzyme or radiolabels, and the like.

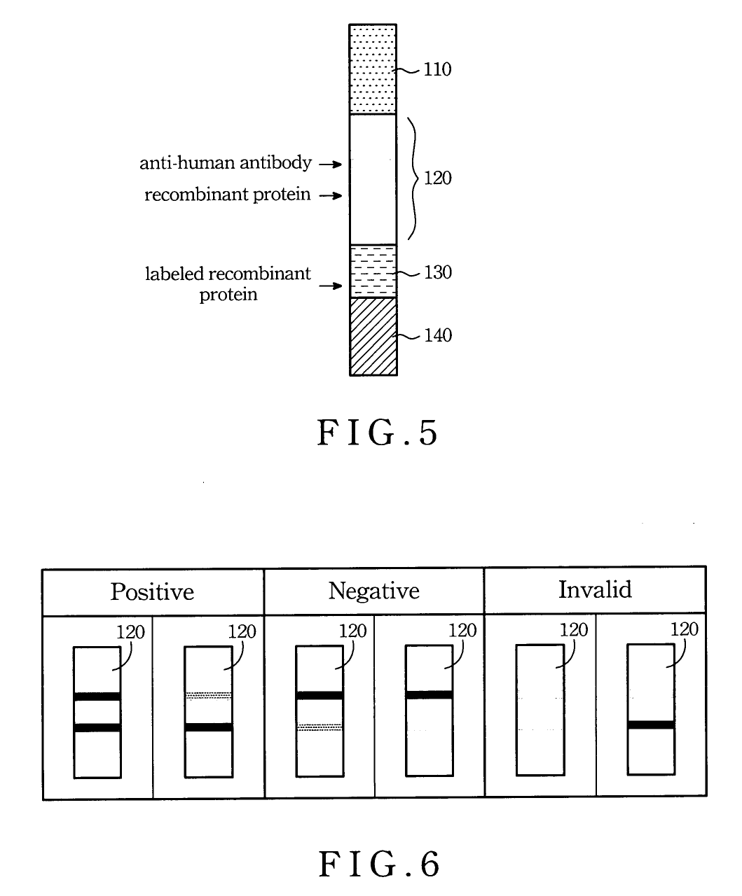

Next, the pellet is resuspended in 10 mM Tris buffer containing 0.1% BSA and having a pH of from 7.5 to 8.5. Referring to FIG. 5, the solution with labeled recombinant spike protein is sprayed on the conjugate pad 130 to a concentration of 20 μl/cm. For long-term preservation, 20% Sucrose (w/v) and 5% (w/v) trehalose are dissolved in the above solution right before the spraying. Thereafter, a drying process is carried out.

II. Method

1. Sample Preparations

A blood sample obtained from a test subject is mixed with 10 mM PBS containing 1% bacterial lysate and having a pH of from 7.2 to 7.6 to a proportion of 1 to 4. In a preferred embodiment of the present invention, the blood sample is selected from the serum.

2. Method of Detecting Anti-SARS Coronavirus Antibody

In one embodiment of the present invention, the sample solution prepared above is dropped onto an area of the sample pad 140. Capillarity allows the sample solution to climb up and react with the recombinant spike protein (antigen) conjugated with gold on the conjugate pad 130. That is, an anti-SARS coronavirus antibody, if existing in the sample solution, will specifically bind to the labeled recombinant antigen on the conjugate pad 130. It is noted that the incubation time should be sufficient to permit antibody-antigen binding to occur, the time being temperature dependent. Suitable incubation times are from 30 to 240 minutes at temperatures within the range of from 16° to 40° C., the preferred contact time being at least 60 minutes at temperatures within the range of from 20° to 26° C.

The sample solution continuously flows throughout the analytical membrane 120. After reaction of the sample suspecting of anti-SARS coronavirus antibody with the recombinant antigen conjugated with gold, the labeled antigen-antibody complex is then reacted with a secondary antigen, such as unconjugated recombinant protein immobilized on the analytical membrane 120, to form a band with a detectable color. The preferred incubation times and temperatures are as set forth above for the binding of the labeled recombinant spike antigen with the test sample anti-SARS coronavirus antibody.

In order to provide an internal control to determine whether the means of detection is available, the amplified fragment obtained from RT-PCR process is integrated into the vector which contains a fragment encoding tag protein. The tag protein is served as a binding target during the purification aforementioned. In one preferred embodiment of the present invention, the RT-PCR product is fused with a gene fragment encoding GST protein in the pGEX-1 vector. Therefore, the targeting recombinant antigen (i.e. spike protein) purified from E. coli carries GST protein.

When the labeled antigen-antibody-antigen binding is forming/formed, unbound recombinant antigen conjugated with gold keeps flowing upwardly to reach and react with the anti-GST antibody to yield a second band with a visible color.

3. Diagnosis of the Detection

Referring to FIG. 6, the color absence of the band of anti-human antibody on the analytical membrane 120 shows that the detecting means is invalid. If the band of anti-human antibody is colorific plus the band of recombinant protein reveals obvious color, the infection of SARS coronavirus is indicated. However, when the band of anti-human antibody appears legible color and the band of recombinant protein is colorless, it exhibits the absence of anti-SARS coronavirus antibody in the test sample.

It should be understood that while the invention has been described in detail herein, the examples were for illustrative purposes only. Other modifications of the embodiments of the present invention that are obvious to those of ordinary skill in the art of molecular biology, medical diagnostics, and related disciplines are intended to be within the scope of the appended claims.

1. A synthetic oligonucleotide primer pair for gene amplification of severe acute respiratory syndrome coronavirus RNA, consisting essentially of a pair of nucleic acid sequences which complement and specifically hybridize to a region of a severe acute respiratory syndrome coronavirus gene consisting of spike protein, wherein said pair of nucleic acid sequences consists of:
SEQ ID NO: 1—TTGGATCCACCGGCCACGGTTTGTGGACCA,
SEQ ID NO: 2—TAGAATTCATTTTGGGTAACTCCAATGCCA, wherein A is adenosine, T is thymidine, G is guanosine and C is cytosine, the sequence being in the 5′ to 3′ orientaton,
and a nucleotide sequence which differs from SEQ ID NO: 1 or SEQ ID NO: 2 by a one base change or substitution therein.

2. A method for detecting the presence or the absence of antibodies against the severe acute respiratory syndrome coronavirus in a test sample, wherein the method comprises the steps of:
immobilizing unlabeled recombinant antigen peptide of SEQ ID NO: 4 on a solid support to form a first area;
immobilizing labeled recombinant antigen peptide of SEQ ID NO: 4 on said solid support to form a second area;
contacting said test sample suspected of containing said antibodies directed against the severe acute respiratory syndrome coronavirus with said labeled recombinant antigen to form labeled antigen/antibody complexes;
reacting said antigen/antibody complexes with said unlabeled recombinant antigen to form labeled sandwich complexes; and
examining the presence of said labeled sandwich complexes at said first area of said solid support, wherein the presence of labeled sandwich complexes at said first area indicates the presence of said antibodies in said test sample.

3. A method of claim 2, further comprising a step of immobilizing an internal control antibody peptide on said solid support to form a third area, said first area being located between said second area and said third area.

4. A method of claim 3, further comprising a step of reacting unbound labeled recombinant antigen with said internal control antibody to examine the validity or invalidity of the support having said unlabeled recombinant antigen, said internal control antibody and said labeled recombinant antigen immobilized thereon.

5. A method of claim 2, wherein said recombinant antigen immobilized on said solid support to form said second area is labeled by colloidal gold.

6. A method of claim 2, wherein said solid support comprises a wicking pad, an analytical membrane, a conjugate pad and a sample pad.

7. A method of claim 2, wherein said test sample is selected from human blood sample.

8. A method of claim 7, wherein said human blood sample comprises human blood serum.

9. A method of claim 4, wherein said labeled recombinant antigen carries a tag protein against said internal control antibody.

10. A method of claim 2, wherein said recombinant antigen is recombinant spike protein.

11. A method of claim 3, wherein said internal control antibody comprises anti-GST antibody.

12. A means for detecting the presence or the absence of severe acute respiratory syndrome coronavirus antibody in a test sample, comprising:
a solid support;
an unlabeled recombinant antigen peptide of SEQ ID NO: 4 being immobilized on a first area of said solid support;
a labeled recombinant antigen peptide of SEQ ID NO: 4 being immobilized on a second area of said solid support; and
an internal control antibody peptide being immobilized on a third area of said solid phase, wherein said first area is located between said second area and said third area.

13. A means of claim 12, wherein said recombinant antigen immobilized on said solid support to form said second area is labeled by colloidal gold.

14. A means of claim 12, wherein said solid support comprises a wicking pad, an analytical membrane, a conjugate pad and a sample pad.

15. A means of claim 12, wherein said test sample is selected from human blood sample.

16. A means of claim 15, wherein said human blood sample comprises human blood serum.

17. A means of claim 12, wherein said labeled recombinant antigen carries a tag protein against said internal control antibody.

18. A means of claim 12, wherein said recombinant antigen is recombinant spike protein.

19. A means of claim 12, wherein said internal control antibody comprises anti-GST antibody.

20. A recombinant protein obtained from the prokaryote transfected by a vector with gene amplification product of claim 1 integrated therein.


SEQUENCE LISTING (4 sequences)

SEQ ID NO: 1 - DNA, 30 bp, Artificial Sequence
  Description of Artificial Sequence Synthetic primer
ttggatccaccggccacggtttgtggacca

SEQ ID NO: 2 - DNA, 30 bp, Artificial Sequence
  Description of Artificial Sequence Synthetic primer
tagaattcattttgggtaactccaatgcca

SEQ ID NO: 3 - DNA, 1173 bp, Human severe acute respiratory syndrome virus
caccggccacggtttgtggaccaaaattatccactgaccttattaagaaccagtgtgtcaattttaattttaatggactcactggtactggtgtgttaactccttcttcaaagagatttcaaccatttcaacaatttggccgtgatgtttctgatttcactgattccgttcgagatcctaaaacatctgaaatattagacatttcaccttgcgcttttgggggtgtaagtgtaattacacctggaacaaatgcttcatctgaagttgctgttctatatcaagatgttaactgcactgatgtttctacagcaattcatgcagatcaactcacaccagcttggcgcatatattctactggaaacaatgtattccagactcaagcaggctgtcttataggagctgagcatgtcgacacttcttatgagtgcgacattcctattggagctggcatttgtgctagttaccatacagtttctttattacgtagtactagccaaaaatctattgtggcttatactatgtctttaggtgctgatagttcaattgcttactctaataacaccattgctatacctactaacttttcaattagcattactacagaagtaatgcctgtttctatggctaaaacctccgtagattgtaatatgtacatctgcggagattctactgaatgtgctaatttgcttctccaatatggtagcttttgcacacaactaaatcgtgcactctcaggtattgctgctgaacaggatcgcaacacacgtgaagtgttcgctcaagtcaaacaaatgtacaaaaccccaactttgaaatattttggtggttttaatttttcacaaatattacctgaccctctaaagccaactaagaggtcttttattgaggacttgctctttaataaggtgacactcgctgatgctggcttcatgaagcaatatggcgaatgcctaggtgatattaatgctagagatctcatttgtgcgcagaagttcaatggacttacagtgttgccacctctgctcactgatgatatgattgctgcctacactgctgctctagttagtggtactgccactgctggatggacatttggtgctggcgctgctcttcaaataccttttgctatgcaaatggcatataggttcaatggcattggagttacccaaaatg

SEQ ID NO: 4 - protein, 390 aa, Human severe acute respiratory syndrome virus
PATVCGPKLSTDLIKNQCVNFNFNGLTGTGVLTPSSKRFQPFQQFGRDVSDFTDSVRDPKTSEILDISPCAFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQN